(19) United States
(12) Patent Application Publication
(10) Pub. No.: US 2015/0086628 A1
(43) Pub. Date: Mar. 26, 2015

(54) ANTIRETROVIRAL COMPOSITION

(71) Applicant: CIPLA LIMITED, Mumbai (IN)

(72) Inventors: Shrinivas Madhukar Purandare, Mumbai (IN); Geena Malhotra, Mumbai (IN)

(21) Appl. No.: 14/398,178

(22) PCT Filed: May 3, 2013
(86) PCT No.: PCT/GB2013/000193

§ 371 Date: Oct. 31, 2014

(30) Foreign Application Priority Data
May 3, 2012 (IN) 1380/MUM/2012
Sep. 6, 2012 (IN) 2590/MUM/2012

(51) Int. Cl. A61K 9/00; A61K 9/16; A61K 9/50; A61K 31/426; A61K 31/513

(52) U.S. Cl. CPC A61K 9/0053; A61K 31/426; A61K 31/513; A61K 9/5073; A61K 9/5089; A61K 9/5084; A61K 9/1611; A61K 9/1635; A61K 9/1623; A61K 9/1617
USPC 424/465; 514/274; 424/490

(57) ABSTRACT

The present invention provides a pharmaceutical solid oral sprinkle composition comprising one or more antiretroviral drugs, and a method of manufacturing the same. The present invention is particularly useful for treatment of an HIV infection, AIDS related complex, or AIDS.

### FIELD OF INVENTION

The present invention relates to a pharmaceutical solid oral sprinkle composition comprising one or more anti-retroviral drugs, such as ritonavir, a manufacturing process thereof, and a use of said composition for the treatment of diseases caused by retroviruses, especially acquired immune deficiency syndrome or an HIV infection. In particular, the present invention relates to a sprinkle formulation comprising one or more anti-retroviral drug, such as ritonavir.

### BACKGROUND AND PRIOR ART

Acquired Immune Deficiency Syndrome (AIDS) causes a gradual breakdown of the body's immune system as well as progressive deterioration of the central and peripheral nervous systems. Two distinct retroviruses, human immunodeficiency virus (HIV) type-1 (HIV-1) or type-2 (HIV-2), have been etiologically linked to the immunosuppressive disease, acquired immunodeficiency syndrome (AIDS). HIV seropositive individuals are initially asymptomatic but typically develop AIDS related complex (ARC) followed by AIDS. Affected individuals exhibit severe immunosuppression, which predisposes them to debilitating and ultimately fatal opportunistic infections. Retroviral replication routinely features post-translational processing of polyproteins. This processing is accomplished by virally encoded HIV protease enzyme. This yields mature polypeptides that will subsequently aid in the formation and function of infectious virus. If this molecular processing is stifled, then the normal production of HIV is terminated. Therefore, inhibitors of HIV protease may function as anti-HIV viral agents.

There are various compositions comprising HIV protease inhibitors and methods of preparing the same.

Ritonavir is chemically designated as 1,3-thiazol-5-ylmethyl N-[(2S,3S,5S)-3-hydroxy-5-[(2S)-3-methyl-2-{[methyl({[2-(propan-2-yl)-1,3-thiazol-4yl]methyl})carbamoyl]amino}butanamido]-1,6-diphenylhexan-2-yl]carbamate and has the following structure.

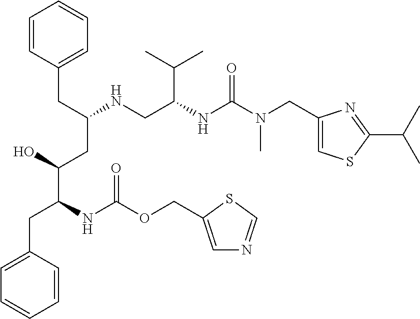 [SMILES: CC(C)c1nc(CN(C)C(=O)N[C@H](CN[C@@H](Cc2ccccc2)C[C@H](O)[C@H](Cc2ccccc2)NC(=O)OCc2cncs2)C(C)C)cs1]

Ritonavir is a protease inhibitor with activity against Human Immunodeficiency Virus Type 1 (HIV-1). Protease inhibitors block the part of HIV called protease. HIV-1 protease is an enzyme required for the proteolytic cleavage of the viral polyprotein precursors into the individual functional proteins found in infectious HIV-1. Ritonavir binds to the protease active site and inhibits the activity of the enzyme. This inhibition prevents cleavage of the viral poly proteins resulting in the formation of immature non-infectious viral particles. A preferred dosage of ritonavir is from about 10 to 200 mg. Further, protease inhibitors are typically used in combination with at least one other anti-HIV drug. Ritonavir is widely given in combination with lopinavir. Ritonavir is commercially available as tablets and oral solution under the trade name NORVIR® in the United States and Europe.

Ritonavir and its salts were first described in U.S. Pat. No. 5,541,206. Said patent describes the structure of ritonavir and the processes for its preparation. Further it describes pharmaceutical compositions and process for making compositions comprising ritonavir. The compositions described are administered orally, parenterally, sublingually, by inhalation spray, rectally, or topically in dosage unit formulations containing conventional nontoxic pharmaceutically acceptable carriers, adjuvants, and vehicles. Solid dosage forms for oral administration include capsules, tablets, pills, powders, and granules. However, said patent does not disclose taste masked compositions of ritonavir.

Lopinavir and its salts are first described in U.S. Pat. No. 5,914,332. Said patent describes the structure of lopinavir and the processes for its preparation. Further it describes pharmaceutical compositions comprising lopinavir. The patent further describes a preferred dosage form as a soft elastic gelatin capsule (SEC) or a hard gelatin capsule. The combination of lopinavir with ritonavir and the use for inhibition or treatment of HIV or AIDS in combination is also described in said patent. Ritonavir on co-administration with lopinavir causes an improvement in the pharmacokinetics (i.e., increases half-life, increases the time to peak plasma concentration, increases blood levels) of lopinavir. However, for specific patient populations such as geriatrics and paediatrics, the dosage form as suggested may be of concern as these patient populations may experience difficulty in swallowing larger sized tablets or capsules, leading to poor patient compliance.

WO9822106 describes a liquid pharmaceutical composition of compounds which are inhibitors of HIV protease with improved oral bioavailability. This application, in particular, describes a composition in the form of a solution which comprises (a) the HIV protease inhibitor, (b) a pharmaceutically acceptable organic solvent and, optionally, (c) a surfactant. It is further described that the composition can be optionally encapsulated in either hard gelatin capsules or soft elastic capsules (SEC). The preferred HIV protease inhibitor is a lopinavir/ritonavir combination. The above process involves a complex manufacturing process. However, said patent does not disclose taste masked sprinkle compositions of ritonavir.

WO02096395 relates to soft elastic capsules and HIV protease inhibiting compounds contained in the soft elastic capsule. The application describes soft elastic capsules that have: a fill, which includes pharmaceutical agents; an alcohol; a fatty acid; and a shell, which includes gelatin and plasticizing agents. It is well known in the art that there is a limited choice of excipients/carriers compatible with gelatin. In general, capsules have crosslinking problems, and to overcome these problems, fillers and stabilizers like citric acid, glycine needs to be incorporated. However, said patent does not disclose taste masked compositions of ritonavir.

WO2008017867 relates to a solid oral composition comprising one or more anti-retroviral drugs, such as lopinavir and ritonavir, with a water insoluble polymer, however, the specific formulation is silent on the dosage forms for specific patient populations such as geriatrics and paediatrics.

WO95/07696 discloses an encapsulated solid or semi-solid dosage form for ritonavir. However, said patent does not disclose taste masked compositions of ritonavir.

For most of the therapeutic agents, to produce systemic effects, the oral route still represents the preferred way of administration, owing to its several advantages and high patient compliance, as compared to any other routes of administration. Tablets and hard gelatin capsules still constitute a major portion of drug delivery systems that are currently available. However, many patient groups such as the elderly, children, and patients who are mentally retarded, uncooperative, nauseated, or on reduced liquid-intake/diets, have difficulties swallowing dosage forms such as tablets and hard gelatin capsules. Further, those who are traveling or have little access to water are similarly affected.

Also, the route of drug administration, appearance, colour, taste, tablet size and dosing regimen are most important parameters that govern patient compliance.

Patient compliance is an important aspect of HIV treatment. If patient compliance decreases, the therapeutic efficiency of the treatment decreases, which in turn may increase resistance to the said treatment. Dosage forms which lead to improved patient compliance therefore improve the overall long term therapeutic efficacy of the treatment. Issues surrounding patient compliance are particularly important for long-term treatments involving chronic infections such as HIV.

In particular, geriatric and paediatric patients often experience difficulty in swallowing larger sized tablets, since large size tablets may result in oesophageal damage due to their physical characteristics, if they are not swallowed properly, which may lead to poor patient compliance.

Also, oral administration of bitter drugs with an acceptable degree of palatability is a key issue for health care providers, especially for paediatric patients. In the case of paediatric and geriatric patients, unpleasant taste should be avoided, since it leads to noncompliance resulting in decreased therapeutic efficacy.

Patients particularly prefer oral dosage forms that are easy to swallow and have a pleasant taste or no taste at all. Objectionable taste is one of the most important formulation problems that are found with certain drugs. This is a distinct problem for drugs which are required to be formulated in an oral dosage form. Thus oral administration of bitter drugs is a major concern for patient compliance.

Further, there has been an enhanced demand for dosage forms that are more patient-friendly and compliant. Since the development cost of a new drug molecule is very high, efforts are now being made to focus on the development of new drug dosage forms for existing drugs with improved safety and efficacy together with reduced dosing frequency as well as which are cost-effective.

Several taste masking techniques are available, such as sensory masking by adding correctives, and chemical masking by chemical modification, such as preparation of inclusion compounds and prodrugs, masking by using a matrix, and physical masking by use of additives. Many techniques have been developed, not only to improve the taste of the molecule but also the formulation and performance of the molecule. These include inclusion complex formation with cyclodextrin, use of ion exchange resin, solubility limiting methods, liposome and multiple emulsions, etc. However all such techniques involve use of complex methods or systems and moreover are expensive.

Hence, to fulfil these medical needs, and to overcome the issues of patient compliance, there remains a need to produce suitable dosage forms which meet the aforementioned requirements.

### OBJECT OF THE INVENTION

An object of the present invention is to provide a pharmaceutical solid oral composition, in the form of a sprinkle formulation, for use with children and other patients, who have difficulty swallowing the conventional solid dosage forms.

Another object of the present invention is to provide a pharmaceutical solid oral composition, in the form of a sprinkle formulation, which may be administered without the need to take it with water.

Another object of the present invention is to provide, a pharmaceutical solid oral composition, in the form of a sprinkle formulation, weighing a minimal amount for use in children and other patients.

Another object of the present invention is to provide a pharmaceutical solid oral composition, in the form of a sprinkle formulation, with a taste masking property and for providing better patient compliance for use with children and other patients.

Another object of the present invention is to provide a pharmaceutical solid oral composition, which is a sprinkle formulation, in the form of powders, powders for reconstitution, pellets, beads, mini-tablets, film coated tablets, film coated tablets MUPS, orally disintegrating MUPS, pills, micro-pellets, small tablet units, MUPS (multiple unit pellet system), disintegrating tablets, dispersible tablets, granules, effervescent granules and microspheres.

Still another object of the present invention is to provide a pharmaceutical solid oral composition, in the form of a sprinkle formulation, which is easy to manufacture.

Another object of the present invention is to provide a process for preparing the pharmaceutical solid oral composition, in the form of a sprinkle formulation.

Yet another object of the present invention is to provide a method of treating diseases caused by retroviruses, especially acquired immune deficiency syndrome or an HIV infection, which method comprises administering the said pharmaceutical solid oral composition, in the form of a sprinkle formulation.

Another object of the present invention is to provide, use of the pharmaceutical solid oral composition, in the manufacture of a medicament for the treatment of an acquired immune deficiency syndrome or in HIV infection, which medicament is in the form of a sprinkle formulation.

### SUMMARY OF THE INVENTION

According to one aspect of the present invention, there is provided a pharmaceutical solid oral composition, in the form of a sprinkle formulation, comprising one or more antiretroviral drugs.

According to an aspect of the present invention, there is provided a pharmaceutical solid oral composition, in the form of a sprinkle formulation, comprising ritonavir.

According to another aspect of the present invention, there is provided a pharmaceutical solid oral composition, in the form of a sprinkle formulation, comprising one or more antiretroviral drugs and at least one polymer.

According to another aspect of the present invention, there is provided a pharmaceutical solid oral composition, in the form of a sprinkle formulation, comprising ritonavir and at least one polymer.

According to yet another aspect of the present invention there is provided a process of manufacturing a pharmaceutical solid oral composition in the form of a sprinkle formulation, comprising one or more antiretroviral drugs and one or more pharmaceutically acceptable excipients.

According to yet another aspect of the present invention there is provided a process of manufacturing a pharmaceutical solid oral composition, which is a sprinkle formulation, comprising a plurality of particles or sub-units, the plurality of particles comprising one or more antiretroviral drugs, a polymer; and optionally one or more pharmaceutically acceptable excipients, comprising hot melt extruding the one or more antiretroviral drug to form an extrudate, then formulating the extrudate into the plurality of particles or sub-units, and combining the plurality of particles or sub-units to provide the solid oral composition.

According to yet another aspect of the present invention there is provided a process of manufacturing a pharmaceutical solid oral composition, which is a sprinkle formulation, comprising a plurality of particles or sub-units, the plurality of particles comprising ritonavir, a polymer and optionally one or more pharmaceutically acceptable excipients, comprising hot melt extruding ritonavir to form an extrudate, then formulating the extrudate into the plurality of particles or sub-units, and combining the plurality of particles or sub-units to provide the solid oral composition.

According to yet another aspect of the present invention there is provided a method of treating diseases caused by retroviruses, especially acquired immune deficiency syndrome or an HIV infection, which method comprises administering pharmaceutical solid oral composition, in the form of sprinkle formulation, comprising one or more antiretroviral drugs.

According to yet another aspect of the present invention there is provided a method of treating diseases caused by retroviruses, especially acquired immune deficiency syndrome or an HIV infection, which method comprises administering a pharmaceutical solid oral composition, in the form of a sprinkle formulation, comprising ritonavir.

According to yet another aspect, the present invention provides a use of the pharmaceutical solid oral composition in the manufacture of a medicament for the treatment of an acquired immune deficiency syndrome or an HIV infection.

### DETAILED DESCRIPTION OF THE INVENTION

The inventors of the present invention have developed a pharmaceutical solid oral composition comprising one or more antiretroviral drugs, and a polymer comprising; a water swellable polymer; a water insoluble polymer; and any combination thereof, wherein the solid oral composition is in the form of a sprinkle formulation, which may be conveniently administered to specific patient populations such as geriatrics and paediatrics.

Accordingly, the pharmaceutical solid oral composition may comprise one antiretroviral drug, preferably ritonavir or two antiretroviral drugs, preferably ritonavir and lopinavir.

As discussed above, the present invention relates to a pharmaceutical solid oral composition comprising one or more antiretroviral drugs and one or more pharmaceutically acceptable excipients, wherein the pharmaceutical solid oral composition may be administered without water or any other suitable liquid.

Further, said pharmaceutical solid oral composition comprises suitable excipients within limited ranges, or minimal amounts, so as to provide a bare minimum weight to the pharmaceutical solid oral composition, which may be achieved by the use of a simple manufacturing process and further exhibits taste masking property along with enhanced bioavailability.

After rigorous experimentation it was surprisingly found that the bitter taste of the one or more antiretroviral drug can be masked by simple and cost-effective process to obtain a taste-masked solid oral composition.

For specific patient populations such as geriatrics and paediatrics, the dosage form as suggested may be of concern as these patient populations may experience difficulty in swallowing larger sized tablets or capsules, leading to poor patient compliance.

The present invention further relates to a pharmaceutical solid oral sprinkle composition comprising one or more antiretroviral drugs and one or more pharmaceutically acceptable excipients, for use in geriatrics and paediatrics.

Thus, the present invention provides a pharmaceutical solid oral sprinkle composition is in the form of a sprinkle formulation comprising one or more antiretroviral drugs and one or more pharmaceutically acceptable excipients, for use in geriatrics and paediatrics.

Further, the inventors have found that when, by a process comprising hot melt extrusion of one or more antiretroviral drugs, preferably ritonavir, with a polymer comprising: a water soluble polymer; a water swellable polymer; a water insoluble polymer; or any combination thereof, the resulting product acquires taste masking property wherein the ratio of drug:polymer is 1:1 to 1:6 by weight. This surprising taste-masked property of the resulting product therefore obviates the need for any other further processing techniques that are used to mask taste, such as addition of flavouring agent, complexation with ion-exchange, microencapsulation, prodrug approach, inclusion complexation, multiple emulsion technique, bitterness inhibitor and providing film and/or seal coatings. Since these techniques would involve additional process steps, and would typically provide further bulk to the composition, the present invention may allow for a simpler manufacturing process and/or reduced bulk of the final pharmaceutical solid oral composition.

According to the present invention, the pharmaceutical solid oral composition may be in the form of a “sprinkle formulation”. The term “sprinkle formulation” as used throughout the specification is a formulation comprising a plurality of particles that can be sprinkled on and mixed with consumable item.

The sprinkle formulation may comprise a plurality of particles or sub-units, which may be provided in a form comprising: a powder; powders for reconstitution; beads; pellets; mini-tablets; film coated tablets; film coated tablets MUPS; orally disintegrating MUPS; pills; micro-pellets; small tablet units; MUPS; disintegrating tablets; dispersible tablets; granules; effervescent granules; microspheres; or any combination thereof. Such particles may be incorporated in capsules or sachets. Preferably, the pharmaceutical solid oral composition according to the present invention is a sprinkle formulation comprising particles in the form of mini-tablets or granules that may be incorporated in a hard gelatin capsule, sachet or packet.

The term “particle” in context of the of the composition of the present invention would be defined as the smallest unit of the composition.

The pharmaceutical solid oral sprinkle composition according to the present invention may comprise one or more antiretroviral drugs and one or more pharmaceutically acceptable excipients, and may comprise a plurality of particles or sub-units which may be provided in a form comprising: powders; powders for reconstitution; beads; pellets; mini-tablets; film coated tablets; film coated tablets MUPS; orally disintegrating MUPS; pills; micro-pellets; small tablet units; MUPS; disintegrating tablets; dispersible tablets; granules; effervescent granules; microspheres; or any combination thereof, that may be directly administered by sprinkling the formulation with regular meals.

Alternatively, pharmaceutical solid oral sprinkle composition according to the present invention may be administered with a liquid or semi-solid beverage, such as a juice or water. Preferably, the solid oral composition according to the present invention may be administered by incorporating into a capsule, sachet or packet and then administered through the oral route.

Thus, as used herein, the term “sprinkle formulation” includes any formulation that is suitable for oral administration, wherein the formulation is sprinkled upon any consumable item.

The pharmaceutical solid oral sprinkle composition according to the present invention may comprise one or more antiretroviral drugs and one or more pharmaceutically acceptable excipients that are incorporated in a hard gelatin capsule, sachet or packet to be administered by sprinkling the formulation onto a regular meal, or to be administered with a liquid or semi-solid beverage, such as fruit juices, water, milk, baby formulas, soft foods, apple sauce, yogurt, and the like.

The pharmaceutical solid oral sprinkle composition according to the present invention may be in the form of mini-tablets or granules that are incorporated in a hard gelatin capsule, a sachet or packet comprising one or more antiretroviral drugs, such as ritonavir, and one or more pharmaceutically acceptable excipients to form a sprinkle composition to be administered by sprinkling the formulation onto a regular meal or to be administered with a liquid or a semi-solid beverage, such as fruit juices, water, milk, baby formulas, soft foods, apple sauce, yogurt, and the like.

Accordingly, when the pharmaceutical solid oral sprinkle composition of the present invention is in the form of a capsule, the said capsule may be swallowed whole, or the capsule may opened and the contents sprinkled onto a regular meal or be administered with a liquid or a semi-solid beverage, such as fruit juices, water, milk, baby formulas, soft foods, apple sauce, yogurt, and the like.

Accordingly, when the pharmaceutical solid oral sprinkle composition of the present invention is in the form of a packet or sachet, the said packet or sachet is typically torn open, thereby allowing the contents to be sprinkled onto a regular meal, or be administered with a liquid or a semi-solid beverage, such as fruit juices, water, milk, baby formulas, soft foods, apple sauce, yogurt, and the like.

Further, the pharmaceutical solid oral sprinkle composition of the present invention may also be provided in the form of kit compositions which has an advantage since the patient always has access to the set of instructions for administration contained in the kit. The inclusion of a set of instructions for administration has been shown to improve patient compliance.

It will be understood that the administration of the pharmaceutical solid oral sprinkle composition of the present invention by means of a kit, with a set of instructions for administration diverting the patient to the correct use of the invention is a desirable, additional feature of this invention.

As used herein, the term “an antiretroviral drug” or “one or more antiretroviral drugs” is used to denote either a single antiretroviral drug, or a combination of two, three, four or more such antiretroviral drugs.

The antiretroviral drug may comprise a protease inhibitor. The protease inhibitor may comprise: lopinavir, saquinavir; ritonavir; nelfinavir; amprenavir; indinavir; nelfinavir; atazanavir; lasinavir; palinavir; tirpranavir; fosamprenavir; darunavir; or any combination thereof.

Accordingly, the pharmaceutical solid oral sprinkle composition may comprise one antiretroviral drug, preferably ritonavir, or two antiretroviral drugs, preferably ritonavir and lopinavir.

However, other class of drugs such as nucleoside and nucleotide reverse transcriptase inhibitors (NRTIs), non-nucleoside reverse transcriptase inhibitors (NNRTIs), integrase inhibitors and maturation inhibitors may be used as the one or more antiretroviral drugs.

Each antiretroviral drug may be disposed in the same particle, or in different particles. Thus, in one aspect, each particle may comprise the first antiretroviral drug, the second antiretroviral drug and the at least one polymer. In another aspect, the first antiretroviral drug and the second antiretroviral drug may be disposed in separate particles. Where the two drugs are found in separate particles, the at least one polymer is disposed in the particle comprising the first antiretroviral drug, the second antiretroviral drug, or both.

In one aspect of the present invention, the pharmaceutical solid oral sprinkle composition comprising ritonavir may be administered in combination with lopinavir.

The compositions of the present invention may comprise a nucleoside reverse transcriptase inhibitor (NRTI). The nucleoside reverse transcriptase inhibitor may comprise: zidovudine; didanosine; stavudine; lamivudine; abacavir; adefovir; lobucavir; entecavir; apricitabine; emtricitabine; zalcitabine; dexelvucitabine; alovudine; amdoxovir; elvucitabine; phosphazid; racivir; stampidine; or any combination thereof.

The compositions of the present invention may further comprise a nucleotide reverse transcriptase inhibitor (NtRTI). The nucleotide reverse transcriptase inhibitor may comprise tenofovir; adefovir; or any combination thereof.

The compositions of the present invention may further comprise a non-nucleotide reverse transcriptase inhibitor (NNRTI). The non-nucleotide reverse transcriptase inhibitor may comprise: nevirapine; rilpiverine; delaviridine; efavirenz; etravirine; or any, combination thereof.

The compositions of the present invention may further comprise an integrase inhibitor. The integrase inhibitor may comprise raltegravir; elvitegravir; or any combinations thereof.

It will be appreciated that whenever the term for a specific drug is used in the specification, for instance, the term “ritonavir”, or “lopinavir”, such a term is used in broad sense to include not only “ritonavir” per se, or “lopinavir” per se, but also their pharmaceutically acceptable salts, pharmaceutically acceptable solvates, pharmaceutically acceptable hydrates, pharmaceutically acceptable esters, pharmaceutically acceptable enantiomers, pharmaceutically acceptable derivatives, pharmaceutically acceptable polymorphs, pharmaceutically acceptable prodrugs, and pharmaceutically acceptable complexes. Thus the meaning of the term “ritonavir”, as used throughout the specification, also includes, for instance, solvates of ritonavir, such as, but not limited to, ritonavir ethanolate solvate, ritonavir formamide solvate and partially desolvated formamide solvate. The terms for the other specific drugs are used similarly in a broad sense.

The pharmaceutical solid oral sprinkle composition may comprise ritonavir in an amount from about 10 mg to about 200 mg. Lopinavir, when present, may be present in an amount from about 40 mg to about 800 mg. These doses of ritonavir and lopinavir are suitable for both paediatric and geriatric patients.

The pharmaceutical solid oral sprinkle composition of the present invention may comprise one or more pharmaceutically acceptable excipients comprising polymers, fillers or diluents, surfactants, solubility enhancers, disintegrants, binders, lubricants, non-ionic solubilisers, glidants, and combinations thereof.

The pharmaceutical solid oral sprinkle composition of the present invention may comprise a water insoluble polymer. The water insoluble polymer may comprise: an acrylic copolymer, e.g. Eudragit E100 (a cationic copolymer based on dimethylaminoethyl methacrylate, butyl methacrylate, and methyl methacrylate), Eudragit EPO (a cationic copolymer based on dimethylaminoethyl methacrylate, basic butylated methacrylate copolymer, butyl methacrylate, and methyl methacrylate), Eudragit L30D-55 (an aqueous dispersion of anionic polymers with methacrylic acid as a functional group), Eudragit FS30D (an aqueous dispersion of an anionic copolymer based on methyl acrylate, methyl methacrylate and methacrylic acid), Eudragit RL30D (a copolymer of ethyl acrylate, methyl methacrylate and a low content of methacrylic acid ester with quaternary ammonium groups), Eudragit RS30D (a copolymer of ethyl acrylate, methyl methacrylate and a low content of methacrylic acid ester with quaternary ammonium groups), Eudragit NE30D (an aqueous dispersion of a neutral copolymer based on ethyl acrylate and methyl methacrylate), or Acryl-Eze; a polyvinylacetate; a cellulose derivative such as ethylcellulose, cellulose acetate Aquacoat ECD (an aqueous dispersion of ethylcellulose (EC) polymer) and Aquacoat CPD (a Cellulose Acetate Phthalate Aqueous Dispersion); or any combinations thereof.

The pharmaceutical solid oral sprinkle composition of the present invention may comprise a water soluble polymer. The water soluble polymer may comprise: copovidone; homopolymer of N-vinyl lactam or copolymer comprising N-vinyl lactam, for instance, homopolymer consisting of or co-polymer comprising N-vinyl pyrrolidine, for instance, polyvinylpyrrolidone (PVP), co-polymer of PVP and vinyl acetate, co-polymer of N-vinyl pyrrolidone and vinyl acetate or vinyl propionate; cellulose ester; cellulose ether; high molecular weight polyalkylene oxide such as polyethylene oxide, polypropylene oxide, or a co-polymer of ethylene oxide and propylene oxide; or any combination thereof.

The pharmaceutical solid oral sprinkle composition of the present invention may comprise a water swellable polymer. The water swellable polymer may comprise: polyethylene oxide; poly (hydroxy alkyl methacrylate); poly (vinyl) alcohol, having low acetal residue and which is cross-linked with glyoxal, formaldehyde or glutaraldehyde and having a degree of polymerization of from 200 to 30,000; mixture of methyl cellulose, cross-linked agar and carboxymethyl cellulose; Carbopol® carbomer which is an acidic carboxy polymer; a Cyanamer® polyacrylamide; cross-linked water swellable indene-maleic anhydride polymer; Goodrich® polyacrylic acid; starch graft copolymer; Aqua Keeps® acrylate polymer polysaccharide comprised of condensed glucose units such as diester cross-linked polyglucan, and the like; Amberlite® ion exchange resin; Explotab® sodium starch glycolate; Ac-Di-Sol® croscarmellose sodium, or any combination thereof.

The pharmaceutical solid oral sprinkle composition of the present invention may further comprise a diluent or filler. The diluent or filler for use in a low dose pharmaceutical composition of the present invention may comprise one or more of: sucrose, calcium silicate; pregelatinized starch; croscarmellose sodium; sodium starch glycolate; lactose; lactose monohydrate (for example, spray-dried lactose, α-lactose, β-lactose); lactose available under the trade mark Tablettose; various grades of lactose available under the trade mark Pharmatose or other commercially available forms of lactose; lactitol; saccharose; sorbitol; mannitol; dextrates; dextrins; dextrose; maltodextrin; croscarmellose sodium; silicified microcrystalline cellulose; microcrystalline cellulose (for example, microcrystalline cellulose available under the trade mark Avicel); hydroxypropylcellulose; L-hydroxypropylcellulose (low substituted); hydroxypropyl methylcellulose (HPMC); methylcellulose polymers (for example, Methocel A, Methocel A4C, Methocel A15C, Methocel A4M); silicified microcrystalline cellulose; hydroxyethylcellulose; sodium carboxymethylcellulose; carboxymethylene; carboxymethyl hydroxyethylcellulose; other cellulose derivatives, starches or modified starches (including potato starch, corn starch, maize starch and rice starch); or any mixture thereof.

The pharmaceutical solid oral sprinkle composition of the present invention may further comprise a binder. The binder may comprise: polyvinyl pyrrolidone (also known as povidone); polyethylene glycol; acacia; alginic acid; agar; calcium carragenan; cellulose derivative such as ethyl cellulose, methyl cellulose, hydroxypropyl cellulose, hydroxypropyl methyl cellulose or sodium carboxymethylcellulose; microcrystalline cellulose; dextrin; gelatin; gum arabic; guar gum; tragacanth; sodium alginate; copovidone; starches; any other pharmaceutically acceptable substances with cohesive properties; or any combination thereof.

The pharmaceutical solid oral sprinkle composition of the present invention may further comprise a disintegrant. The disintegrant may comprise: crospovidone; ac-di-sol; sodium starch glycolate; hydroxylpropyl cellulose (HPC); low density HPC; carboxymethylcellulose (CMC); sodium CMC; calcium CMC; croscarmellose sodium; carboxymethyl starch; hydroxylpropyl starch; modified starch; crystalline cellulose; sodium starch glycolate; alginic acid or a salt thereof, such as sodium alginate; or any combination thereof.

The pharmaceutical solid oral sprinkle composition of the present invention may further comprise a solubility enhancer. The solubility enhancer may comprise: stearoyl macrogol glyceride; sorbitan monolaurate (Span 20); Polyoxyl castor oil; or any combination thereof.

The pharmaceutical solid oral sprinkle composition of the present invention may further comprise a lubricant, glidant and/or an anti-adherent. The glidant, anti-adherents and/or lubricant may comprise: stearic acid and pharmaceutically acceptable salts or esters thereof (for example, magnesium stearate, calcium stearate, sodium stearyl fumarate or other metallic stearate); talc; waxes (for example, microcrystalline waxes) and glycerides; light mineral oil; PEG; silica acid or a derivative or salt thereof (for example, silicates, silicon dioxide, colloidal silicon dioxide and polymers thereof, crospovidone, magnesium stearate, magnesium aluminosilicate and/or magnesium alumino metasilicate); sucrose ester of fatty acids; hydrogenated vegetable oils (for example, hydrogenated castor oil); or any mixture thereof.

The pharmaceutical solid oral sprinkle composition of the present invention may further comprise a preservative. The preservative may comprise: benzoic acid; sorbic acid; butylparaben; ethylparaben; methylparaben; propylparaben; sodium benzoate; sodium; propionate; or any combination thereof.

The pharmaceutical solid oral sprinkle composition of the present invention may further comprise a sweetener. The sweetener may comprise: saccharin sodium, aspartame, acesulfame, cyclamate, alitame, a dihydrochalcone sweetener, monellin, neohesperidin, neotame, stevioside, sucralose, any pharmaceutically acceptable salts thereof, and the like, or combination thereof.

The pharmaceutical solid oral sprinkle composition of the present invention may be administered orally through known solid dosage forms.

The pharmaceutical solid oral sprinkle composition may be provided in the form of a sprinkle formulation. In other words, the pharmaceutical solid oral composition is in a form suitable for administration by sprinkling onto a consumable item. The sprinkle formulation may comprise a plurality of particles or sub-units, which may be provided in a form comprising: a powder; powders for reconstitution; a pellet; a bead; a mini-tablet; a pills; a micro-pellet; a small tablet unit; a MUPS; film coated tablets, film coated tablets MUPS, orally disintegrating MUPS a disintegrating tablet; a dispersible tablet; a granule; an effervescent granules; a microsphere; or any combination thereof. More preferably, the pharmaceutical solid oral composition is in the form of mini-tablets or granules.

Accordingly, when a patient intakes the pharmaceutical solid oral sprinkle composition sprinkled onto a consumable item, it is preferable that the patient does not chew or crush the composition; instead, the composition should be preferably swallowed whole with the consumable item.

According to the generality of the concept, it is thought that large particle sizes trigger a chewing reflex in a patient, and thus the size of the plurality of particles or sub-units of the pharmaceutical solid oral composition is preferably small enough to prevent a patient's chewing reflex. In other words, it is thought that a patient will find it easier to ingest a consumable item onto which the pharmaceutical solid oral composition of the present invention has been sprinkled, without chewing, if the diameter of the plurality of particles or sub-units is small. Thus, in an aspect, the upper limit of the median or average diameter of the particles of the sprinkle formulation may be less than 2.8 mm, preferably less than 2 mm, more preferably less than 1.5 mm, and most preferably less than 1 mm. The lower limit of the median or average diameter of the particles may be greater than 0.2 mm, most preferably 0.5 mm. A preferred range for the median or average particle diameter is 0.2 mm to 2.8 mm.

The plurality of particles or sub-units of the pharmaceutical solid oral sprinkle composition of the present invention may be enclosed in a hard gelatin capsule, sachet or packet. The capsule may be swallowed whole or opened. The sachet or packet may be torn open, and its contents sprinkled onto consumable item, prior to administration. Preferably, the pharmaceutical solid oral composition of present invention comprises mini-tablets or granules filled in a hard gelatin capsule, a sachet or a packet.

Preferably, the plurality of particles or sub-units are directly administered by sprinkling them on a regular meal, which may then be consumed normally by the patient, for ease of administration. Alternatively, the plurality of particles or sub-units may be administered by sprinkling them into a liquid or semi-solid beverage, such as fruit juices, water, milk, baby formulas, soft foods, apple sauce, or yogurt, and the like, which may then be consumed normally by the patient.

The plurality of particles or sub-units of the present invention may also optionally be coated. Preferably, the plurality of particles or sub-units may be film coated. More preferably, the plurality of particles or sub-units may be seal coated and then film coated. Alternatively, the particles may be film coated and then seal coated.

Such coats have a number of advantages, preventing the one or more antiretroviral drugs from being released into, or interacting with, the consumable item onto which it is sprinkled, and these benefits make coated particulates provide a further advantage of the compositions of the present invention.

Additional excipients such as film forming polymers, solvents, plasticizers, anti-adherents, opacifiers, colorants, pigments, antifoaming agents, and polishing agents can be used in coatings.

Suitable seal forming material may comprise: hydroxypropylmethylcellulose (optionally HPMC 6 CPS, or HPMC 6 CPS to HPMC 15CPS grade); hydroxypropylcellulose; polyvinylpyrrolidone; methylcellulose; carboxymethylcellulose; hypromellose; acacia; gelatin; or any combination thereof, to increase adherence and coherence of the seal coat. Preferably the seal coat comprises hydroxypropylmethylcellulose.

The HPMC component of the seal coating, if present, may be mixed with a solvent, wherein said solvent may comprise: acetone; methylene chloride; isopropyl alcohol; or any combination thereof. The seal coating may also comprise talc.

Suitable film-forming agents include, but are not limited to, cellulose derivatives, such as, soluble alkyl- or hydroalkyl-cellulose derivatives such as methylcelluloses, hydroxymethyl celluloses, hydroxyethyl celluloses, hydroxypropyl celluloses, hydroxymethylethyl celluloses, hydroxypropyl methylcelluloses, sodium carboxymethyl celluloses, insoluble cellulose derivatives such as ethylcelluloses and the like, dextrins, starches and starch derivatives, polymers based on carbohydrates and derivatives thereof, natural gums such as gum Arabic, xanthans, alginates, polyacrylic acids, polyvinyl alcohols, polyvinyl acetates, polyvinylpyrrolidones, polymethacrylates and derivatives thereof, chitosan and derivatives thereof, shellac and derivatives thereof, waxes, fat substances and any mixtures or combinations thereof.

Suitable enteric coating materials, include, but are not limited to, cellulosic polymers like cellulose acetate phthalates, cellulose acetate trimellitates, hydroxypropyl methylcellulose phthalates, polyvinyl acetate phthalates, methacrylic acid polymers, any copolymer thereof, any mixture thereof, or combination thereof.

Some of the excipients are used as adjuvant to the coating process, including excipients such as plasticizers, opacifiers, antiadhesives, polishing agents, and the like.

Suitable plasticizers include, but are not limited to, stearic acid, castor oil, diacetylated monoglycerides, dibutyl sebacate, diethyl phthalate, glycerin, polyethylene glycols, propylene glycols, triacetin, triethyl citrate, or mixtures thereof.

Suitable opacifiers include, but are not limited to, titanium dioxide.

Suitable anti-adhesives include, but are not limited to, talc.

Suitable polishing agents include, but are not limited to, polyethylene glycols of various molecular weights or mixtures thereof, talc, surfactants (glycerol monostearate and poloxamers), fatty alcohols (stearyl alcohol, cetyl alcohol, lauryl alcohol and myristyl alcohol) and waxes (carnauba wax, candelilla wax and white wax), or mixtures thereof.

Suitable solvents used in the processes of preparing the pharmaceutical solid oral composition of the present invention, include, but are not limited to, water, methanol, ethanol, acidified ethanol, acetone, diacetone, polyols, polyethers, oils, esters, alkyl ketones, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethylsulphoxide, N,N-dimethylformamide, tetrahydrofuran, or mixtures thereof.

According to another aspect of the present invention, the solid oral sprinkle composition may be seal coated followed by film coating.

The present invention may be manufactured through various techniques or processes including melt granulation, melt extrusion, spray drying, solution evaporation, direct blending, direct compression, wet granulation, dry granulation, melt lyophilisation, hot melt extrusion, extrusion-spheronization and the like, or combinations thereof. More preferably, the pharmaceutical solid oral composition of the present invention may be manufactured by melt extrusion.

According to one aspect the present invention, there is provided a process for preparing a pharmaceutical solid oral sprinkle composition comprising one or more antiretroviral drugs, such as ritonavir, the process comprising melt extruding comprising the steps: (a) preparing a homogeneous melt of the one or more antiretroviral drugs; a polymer comprising: a water soluble polymer; a water swellable polymer; a water insoluble polymer; or any combination thereof, and optionally one or more excipients; (b) cooling the melt obtained in step (a); (c) allowing the cooled melt to solidify to obtain an extrudate; and (d) processing the extrudate into a desired shape.

Optionally, step (a) is carried out at a temperature ranging from about 70° C. to about 200° C. typically about at a temperature ranging from about 90° C. to about 150° C.

Step (d) may comprise shaping the extrudate into a mini-tablet or granule. Alternatively, step (d) may comprise cutting the extrudate into pieces and further processing the cut extrudate into a suitable dosage form. Alternatively, step (d) may comprise milling and grinding the extrudate to form granules.

Alternatively, the process for manufacturing the pharmaceutical solid oral sprinkle composition in the form of a mini-tablet, which process further comprises step (e) drying and lubricating the granules and compressing the lubricated dried granules to form the mini-tablet. Alternatively, the process may further comprise step (f) seal coating the mini-tablet or granule, or film coating the mini-tablet or granule. Alternatively, the process may further comprise step (f) seal coating the mini-tablet or granule; and step (g) comprising film coating the seal coated mini-tablet or granule.

The seal coat material may be hydroxypropylmethylcellulose. Typically, the hydroxypropylmethylcellulose is hydroxypropylmethylcellulose comprising: hydroxypropyl methylcellulose (HPMC) 6CPS to hydroxypropyl methylcellulose (HPMC) 15CPS.

As mentioned above, the present invention may be manufactured through various techniques.

According to a further aspect of the present invention, there is provided a process for preparing a pharmaceutical solid oral sprinkle composition comprising one or more antiretroviral drugs, such as ritonavir, the process comprising: (a) melt granulating one or more solubility enhancers and one or more first pharmaceutically acceptable excipients with the one or more antiretroviral drugs in water or any other suitable solvent to form a granulated material; (b) sieving the granulated material; (c) drying the sieved granulated material to form dried granules; (d) lubricating the dried granules with one or more lubricants and optionally one or more other pharmaceutically acceptable excipients; and (e) optionally further processing the lubricated dried granules.

The present invention further provides a process for manufacturing a pharmaceutical solid oral sprinkle composition, which process comprises: (1) coating one or more antiretroviral drugs, such as ritonavir, with a polymer comprising: a water soluble polymer; a water swellable polymer; a water insoluble polymer; or any combination thereof, to form coated granules containing one or more antiretroviral drugs; (2) mixing the coated granules obtained in step (1) with one or more pharmaceutically acceptable excipients; and (3)(i) filling the mixture formed in step (2) into a hard gelatin capsule, sachet or packet which may be suitable for sprinkling onto any consumable item by a patient for ease of administration, or (ii) compressing the mixture formed in step (2) to form mini-tablets which optionally may be filled into a capsule, sachet or packet.

Accordingly, the inventors have surprisingly found that when, by a process comprising hot melt extrusion of one or more antiretroviral drugs with a polymer comprising: a water soluble polymer; a water swellable polymer; a water insoluble polymer; and any combination thereof, the resulting product acquires taste masking property wherein the ratio of drug:polymer is 1:1 to 1:6.

It was surprisingly found that, while carrying out the melt extrusion process, an in-situ reaction occurred between the drug and the polymer. This in-situ reaction led to an ionic interaction between the drug and the polymer eventually leading to taste masked product.

In general terms, the process of hot melt extrusion is carried out in conventional extruders known to a person skilled in the art. The melt-extrusion process comprises the steps of preparing a homogeneous melt of the one or more antiretroviral drugs, the polymer and any the excipients, if present, and cooling the melt until it solidifies. “Melting” means a transition into a liquid or rubbery state in which it is possible for one component to become embedded homogeneously in the other. Typically, one component will melt and the other components will dissolve in the melted component, thus forming a solution. Melting usually involves heating above the softening point of the polymer. The preparation of the melt can take place in a variety of ways. The mixing of the components can take place before, during or after the formation of the melt. For example, the components can be mixed first and then melted, or be simultaneously mixed and melted. Usually, the melt is homogenized in order to disperse the active ingredients efficiently. Also, it may be convenient first to melt the polymer and then to mix in and homogenize the active ingredients.

The formation of the extrudate leads to a further advantage, such that the homogenous melt of the one or more antiretroviral drug in the polymer converts the drug into its amorphous form. Drugs thus converted into its amorphous form may exhibit improved bioavailability as compared to its crystalline forms. This is particularly advantageous for drugs whose crystalline forms exhibit poor bioavailability, such as ritonavir.

Usually, the melt temperature is in the range of about 70° C. to about 200° C., preferably from about 80° C. to about 180° C., and most preferably from about 90° C. to about 150° C.

Suitable extruders include single screw extruders, intermeshing screw extruders or else multiscrew extruders, preferably twin screw extruders, which can be co-rotating or counter-rotating and, optionally, be equipped with kneading disks. It will be appreciated that the working temperatures will also be determined by the kind of extruder or the kind of configuration within the extruder that is used.

The extrudates can be in the form of beads, granulates, tube, strand or cylinder, and these can be further processed into any desired shape.

The term ‘extrudates’ as used herein refers to solid product solutions, solid dispersions and glass solutions of one or more drugs in one or more polymers comprising: a water soluble polymer; a water swellable polymer; a water insoluble polymer; or any combination thereof, and optionally pharmaceutically acceptable excipients. Preferably, a powder blend of the one or more antiretroviral drugs, such as ritonavir, the one or more polymer and optionally pharmaceutical excipients are transferred by a rotating screw of a single screw extruder through the heated barrel of an extruder, whereby the powder blend melts, and a molten solution product is collected on a conveyor, where it is allowed to cool to form an extrudate. Shaping of the extrudate may be conveniently be carried out by a calendar with two counter-rotating rollers with mutually matching depressions on their surface. A broad range of tablet forms may be attained by using rollers with different forms of depressions. Alternatively, the extrudate may be cut into pieces after solidification and further processed into suitable dosage forms. More preferably, the extrudates thus obtained from the above process may be then milled and ground to granules by means known to a person skilled in the art.

Further, hot melt extrusion is a fast, continuous manufacturing process without requirement of further drying or discontinuous process steps; it provides short thermal exposure of active allows processing of heat sensitive actives; the process temperatures can be reduced by addition of plasticizers; and comparatively lower investment is required for the equipment as compared to other processes. The entire process may be anhydrous and the intense mixing and agitation of the powder blend that occur during processing contributes to a very homogenous extrudate.

In one aspect, the present invention provides a pharmaceutical solid oral sprinkle composition comprising one or more antiretroviral drugs, such as ritonavir alone, or a combination comprising ritonavir and lopinavir, and a polymer comprising: a water soluble polymer; a water swellable polymer; a water insoluble polymer; and any combination thereof, which are melt extruded by any process as described herein, where the powder blend comprises the one or more antiretroviral drugs, the one or more polymers, and optionally an excipient which may comprise a bulking agent and/or a flavourant. These are so processed to form a powder blend which may be transferred through the heated barrel of the extruder, most preferably single screw extruder, whereby the powder blend melts and molten solution product may be collected on a conveyor whereby it is allowed to cool and form an extrudate. Alternatively, the extrudate is cut into pieces after solidification and may be further processed into suitable dosage forms. More preferably the extrudates thus obtained from the above process may be then milled and ground to granules by means known to a person skilled in the art.

In another aspect, the present invention provides a pharmaceutical solid oral sprinkle composition comprising one or more antiretroviral drugs and a combination of polymers comprising: a water soluble polymer and a water insoluble polymer; a water soluble polymer and a water swellable polymer; a water insoluble polymer and a water swellable polymer; or a water swellable polymer, a water soluble and a water insoluble polymer, which are melt extruded by any process as described herein, where the powder blend comprises one or more antiretroviral drugs, preferably ritonavir alone, or a combination of ritonavir and lopinavir, and at least one polymer, and optionally further comprising at least one or more excipients.

These are so processed to form a powder blend which may be transferred through the heated barrel of the extruder, whereby the powder blend melts and molten solution product is collected on a conveyor whereby it is allowed to cool and form an extrudate. Alternatively, the extrudate may be cut into pieces after solidification and further processed into suitable dosage forms. More preferably the extrudates thus finally obtained from the above process are then milled and ground to granules by means known to a person skilled in the art.

The pharmaceutical solid oral sprinkle composition of the present invention may further comprise a plasticizer. The plasticizer may be incorporated into the composition, depending on the polymer and the process requirement. The plasticizer, advantageously, when used in the hot melt extrusion process, may decrease the glass transition temperature of the polymer. The plasticizer may also help in reducing the viscosity of the polymer melt and thereby allow for lower processing temperature and extruder torque during hot melt extrusion. The plasticizer may comprise: sorbitan monolaurate (Span 20); sorbitan monopalmitate; sorbitan monostearate; sorbitan monoisostearate; a citrate ester type plasticizer, such as triethyl citrate or citrate phthalate; propylene glycol; glycerin; low molecular weight polyethylene glycol; triacetin; dibutyl sebacate; tributyl sebacate; dibutyltartrate; dibutyl phthalate and the like; or any combination thereof. The plasticizer may be present in an amount ranging from 0% to 10% by weight of polymer.

In one aspect, the present invention may be formulated for paediatric patients. From the point of view of paediatric patient acceptability, the bulking agent, when present in the pharmaceutical solid oral composition, may comprise: a saccharide, such as a monosaccharide, a disaccharide, a polysaccharide and the like, or any combinations thereof; a sugar alcohol, such as arabinose, lactose, dextrose, sucrose, fructose, maltose, mannitol, erythritol, sorbitol, xylitol, lactitol and the like, or any combination thereof; or a combination of a saccharide and a sugar alcohol. Alternatively, the bulking agent may comprise: powdered cellulose; a microcrystalline cellulose; a purified sugar; a sugar derivative; or any combination thereof. Most preferably, the bulking agent comprises purified sugar.

Accordingly, the pharmaceutical solid oral sprinkle composition of the present invention may further incorporate pharmaceutically acceptable flavourants. The pharmaceutically acceptable flavourants may comprise: citric acid; tartaric acid; lactic acid; a natural flavourant and the like; or any combination thereof.

In a further aspect, the pharmaceutical solid oral sprinkle composition according to the present invention may also comprise the one or more antiretroviral drugs in nano-size form. Preferably, the active pharmaceutical ingredients have average or median particle size less than about 2000 nm, preferably less than about 1000 nm, more preferably less than 800 nm, and most preferably less than 500 nm. The average or median particle size is greater than 50 nm, more preferably greater than 100 nm, most preferably greater than 200 nm

Nanonization of hydrophobic or poorly water-soluble drugs generally involves the production of drug nano crystals through either chemical precipitation (bottom-up technology) or disintegration (top-down technology). Different methods may be utilized to reduce the particle size of the hydrophobic or poorly water soluble drugs. [Huabing Chen et al., discusses the various methods to develop nano-formulations in “Nanonization strategies for poorly water-soluble drugs,” Drug Discovery Today, Volume 00, Number 00, March 2010]

Nano-sizing leads to increase in the exposure of surface area of particles leading to an increase in the rate of dissolution.

The nanoparticles of the present invention may be obtained by any of the process such as, but not limited to: milling; precipitation; homogenization; high pressure homogenization; spray-freeze drying; use of supercritical fluid technology; the double emulsion/solvent evaporation technique; use of PRINT technology; thermal condensation; ultra-sonication; or any combination thereof.

Accordingly, the process of milling may comprise dispersing drug particles in a liquid dispersion medium in which the drug is poorly soluble, followed by applying mechanical means in the presence of grinding media to reduce the particle size of drug to the desired effective average particle size.

Accordingly, the process of precipitation may involve the formation of crystalline or semi-crystalline drug nanoparticles by nucleation and the growth of drug crystals. In a typical procedure, drug molecules are first dissolved in an appropriate organic solvent such as acetone, tetrahydrofuran or N-methyl-2-pyrrolidone at a super saturation concentration to allow for the nucleation of drug seeds. Drug nano-crystals are then formed by adding the organic mixture to an antisolvent, such as water, in the presence of a stabilizer, such a surfactant. The choice of solvent, stabilizer and the mixing process are key factors to control the size and stability of the drug nano-crystals.

Accordingly, the process of homogenization may involve passing a suspension of crystalline drug and a stabilizer through the narrow gap of a homogenizer at high pressure (which may fall within the range of 500-2000 bar). The pressure creates powerful disruptive forces such as cavitation, collision and shearing, which may disintegrate coarse particles to nanoparticles.

Accordingly, the process of high pressure homogenization may comprise drug pre-suspension (containing drug particles in the micrometer range) by subjecting the drug to air jet milling in the presence of an aqueous surfactant solution. The pre-suspension may then be subjected to high-pressure homogenization in which it passes through a very small homogenizer gap of around 25 μm, which leads to a high streaming velocity. High-pressure homogenization is based on the principle of cavitations (i.e. the formation, growth, and implosive collapse of vapor bubbles in a liquid).

Accordingly, the process of spray-freeze drying involves the atomization of an aqueous drug solution into a spray chamber filled with a cryogenic liquid (liquid nitrogen) or halocarbon refrigerant, such as a chlorofluorocarbon or a fluorocarbon. The water is removed by sublimation after the liquid droplets solidify.

Accordingly, the process of supercritical fluid technology involves controlled crystallization of drug from dispersion in super critical fluids, such as carbon dioxide.

Accordingly, the process of double emulsion/solvent evaporation technique may involve preparation of an oil/water (o/w) emulsion with subsequent removal of the oil phase through evaporation. The emulsion may be prepared by emulsifying the organic phase containing drug, polymer and organic solvent in an aqueous solution containing an emulsifier. The organic solvent diffuses out of the polymer phase and into the aqueous phase, and is then evaporated, forming drug-loaded polymeric nano-particles.

Accordingly, the process of PRINT (Particle replication in non-wetting templates) may involve utilization of a low surface energy fluoro polymeric mould that enables high-resolution imprint lithography, to fabricate a variety of organic particles. PRINT can precisely manipulate particle size of drug ranging from 20 nm to more than 100 nm.

Accordingly, the process of thermal condensation may involve the use of a capillary aerosol generator (CAG) to produce high concentration condensation submicron to micron sized aerosols from drug solutions.

Accordingly, the process of ultra-sonication involves application of ultrasound during particle synthesis or precipitation, which leads to smaller particles of drug and increased size uniformity.

Accordingly, the process of spray drying may involve supplying a feed solution at room temperature and pumping it through a nozzle where it is atomized by the nozzle gas. The atomized solution is then dried by preheated drying gas in a special chamber to remove water moisture from the system, thus forming dry particles of drug.

In a preferred aspect of the present invention, the nano-milled one or more antiretroviral drugs may be obtained by nano-milling of the one or more antiretroviral drugs with at least one surface stabilizer, at least one viscosity building agent and at least one polymer.

The pharmaceutical solid oral sprinkle composition of the present invention can be manufactured by any of the processes as described above.

The present invention also provides a method of treating diseases caused by retroviruses, especially acquired immune deficiency syndrome or an HIV infection, which method comprises administering a pharmaceutical solid oral composition.

The present invention also provides a use of a pharmaceutical solid oral sprinkle composition in the manufacture of a medicament for the treatment of an acquired immune deficiency syndrome or an HIV infection of a patient.

The following examples are for the purpose of illustration of the invention only and are not intended in any way to limit the scope of the present invention.

### Example 1

| Sr. No. | Ingredients | Qty mg/Capsule |
|---|---|---|
| | Dry Mix | |
| 1. | Lopinavir | 40 |
| 2. | Ritonavir | 10 |
| 3. | Colloidal Silicon Dioxide | 2 |
| 4. | Crospovidone | 140.7 |
| 5. | Sorbitan Monolaurate | 14 |
| | Blending & Lubrication | |
| 6. | Colloidal Silicon Dioxide | 2 |
| 7. | Sodium Stearyl Fumarate | 2 |
| | Coating | |
| 8. | Hydroxy Propyl Methyl Cellulose | 12.642 |
| 9. | Polyethylene Glycol 6000 | 0.129 |
| 10. | Talc | 0.129 |
| 11. | Acetone | q.s. |
| 12. | Water | q.s |
| | Total | 223.6 |

### Process:

(1) A dry mix of lopinavir, ritonavir, colloidal silicon dioxide, was prepared.

(2) Sorbitan monolaurate was added over Copovidone in a suitable granulator separately to form polymer premix.

(3) The dry mix obtained in step (1) and step (2) was mixed in a suitable granulator followed by melt extrusion (hot).

(4) Colloidal silicon dioxide was blended with the dried granules and lubricated by using sodium stearyl fumarate.

(5) The lubricated granules were compressed into mini-tablets.

(6) The compressed mini-tablets were coated with seal coating solution.

(7) The mini-tablets obtained in step (6) were filled into hard gelatin capsules.

### Example 2

| Sr. No. | Ingredients | Mg/Sachet |
|---|---|---|
| I. | Dry Mix for Hot melt | |
| | Extrusion | |
| 1. | Ritonavir | 10.00 |
| 2. | Eudragit | 5-50 |
| 3. | Colloidal silicon dioxide | q.s |
| II. | Blending | |
| 4. | Sugar (Pulvarised) | 5-50 |
| 5. | Flavour | q.s |
| 6. | Colloidal silicon dioxide | q.s |
| III. | Average Weight (Range) | 20-110 |

### Process:

1. Dry mix of ritonavir, colloidal silicon dioxide and eudragit was prepared.
2. Dry mix obtained in step (1) was extruded using hot melt extrusion technique.
3. Extrudes obtained in step (2) were sized and sifted to form granules.
4. Granules obtained in step (3) were blended with colloidal silicon dioxide and sugar.

### Example 3

| Sr. No. | Ingredients | Mg/Sachet |
|---|---|---|
| I. | Dry Mix for Hot melt | |
| | Extrusion | |
| 1. | Ritonavir | 10.00 |
| 2. | Eudragit | 5-50 |
| 3. | Sugar (Pulvarised) | 5-50 |
| 4. | Colloidal silicon dioxide | q.s |
| II. | Blending | |
| 5. | Flavour | q.s |
| 6. | Colloidal silicon dioxide | q.s |
| III. | Average Weight (Range) | 20-110 |

### Process:

1. Dry mix of ritonavir, colloidal silicon dioxide, sugar and eudragit was prepared.
2. Dry mix obtained in step (1) was extruded using hot melt extrusion technique.
3. Extrudes obtained in step (2) were sized and sifted to form granules.
4. Granules obtained in step (3) were blended adding colloidal silicon dioxide.

### Example 4

| Sr. No. | Ingredients | Mg/Capsule |
|---|---|---|
| I. | Dry Mix for Hot melt Extrusion | |
| 1. | Ritonavir | 30.00 |
| 2. | Basic butylated methacrylate copolymer | 15-45 |
| 3. | Sucrose | 45-135 |
| 4. | Saccharin Sodium | 0.90-2.70 |
| 5. | Colloidal silicon dioxide | 0.60-1.80 |
| 6. | Stearic acid | 2.50-6.50 |
| II. | Blending | |
| 7. | Colloidal silicon dioxide | 1.0-3.0 |
| III. | Total Weight | 95-224 |

### Process:

1. Dry mix of ritonavir, colloidal silicon dioxide, sucrose, saccharin sodium, basic butylated methacrylate copolymer and stearic acid was prepared.
2. Dry mix obtained in step (1) was extruded using hot melt extrusion technique.
3. Extrudes obtained in step (2) were sized and sifted to form granules.
4. Granules obtained in step (3) were blended adding colloidal silicon dioxide.
5. The blended granules obtained in step (4) were filled in capsules.

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to fall within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise. Thus, for example, reference to “an excipient” includes a single excipient as well as two or more different excipients, and the like.

It will be appreciated that the invention may be modified within the scope of the claims which follow.

1. A pharmaceutical solid oral sprinkle composition, comprising a plurality of particles, the plurality of particles comprising a first and a second antiretroviral drug and at least one polymer, wherein the first antiretroviral drug comprises ritonavir.

2. A pharmaceutical solid oral sprinkle composition according to claim 1, wherein the ritonavir is provided as a pharmaceutically acceptable salt, pharmaceutically acceptable solvate, pharmaceutically acceptable hydrate, pharmaceutically acceptable ester, pharmaceutically acceptable enantiomer, pharmaceutically acceptable derivative, pharmaceutically acceptable polymorph, pharmaceutically acceptable prodrug, or pharmaceutically acceptable complex thereof.

3. A pharmaceutical solid oral sprinkle composition according to claim 1 wherein the ritonavir is provided as a pharmaceutically acceptable solvate thereof.

4. A pharmaceutical solid oral sprinkle composition according to claim 1 wherein the ritonavir is provided as its ethanolate solvate, formamide solvate or partially desolvated formamide solvate.

5. A pharmaceutical solid oral sprinkle composition according to claim 1, wherein the second antiretroviral drug comprises a protease inhibitor; a nucleoside reverse transcriptase inhibitor; a nucleotide reverse transcriptase inhibitor; a non-nucleoside reverse transcriptase inhibitor; an integrase inhibitor; a maturation inhibitor; or any combination thereof.

6. A pharmaceutical solid oral sprinkle composition according to claim 5, wherein the protease inhibitor comprises saquinavir; nelfinavir; amprenavir; lopinavir, indinavir; nelfinavir; atazanavir; lasinavir; palinavir; tirpranavir; fosamprenavir; darunavir or any combination thereof.

7. A pharmaceutical solid oral sprinkle composition according to claim 5, wherein nucleoside reverse transcriptase inhibitor comprises zidovudine; didanosine; stavudine; lamivudine; abacavir; adefovir; lobucavir; entecavir; apricitabine; emtricitabine; zalcitabine; dexelvucitabine; alovudine; amdoxovir; elvucitabine; phosphazid; racivir; stampidine; or any combination thereof.

8. A pharmaceutical solid oral sprinkle composition according to claim 5, wherein nucleotide reverse transcriptase inhibitor comprises tenofovir and/or adefovir.

9. A pharmaceutical solid oral sprinkle composition according to claim 5, wherein the non-nucleotide reverse transcriptase inhibitor comprises nevirapine; rilpiverine; delaviridine; efavirenz; etravirine; or any combination thereof.

10. A pharmaceutical solid oral sprinkle composition according to claim 5, wherein the integrase inhibitor comprises raltegravir and/or elvitegravir.

11. A pharmaceutical solid oral sprinkle composition according to claim 6, wherein the saquinavir; nelfinavir; amprenavir; lopinavir, indinavir; nelfinavir; atazanavir; lasinavir; palinavir; tirpranavir; fosamprenavir; darunavir; zidovudine; didanosine; stavudine; lamivudine; abacavir; adefovir; lobucavir; entecavir; apricitabine; emtricitabine; zalcitabine; dexelvucitabine; alovudine; amdoxovir; elvucitabine; phosphazid; racivir; stampidine; tenofovir; adefovir; nevirapine; rilpiverine; delaviridine; efavirenz; etravirine; raltegravir or elvitegravir is provided as a pharmaceutically acceptable salt, pharmaceutically acceptable solvate, pharmaceutically acceptable hydrate, pharmaceutically acceptable ester, pharmaceutically acceptable enantiomer, pharmaceutically acceptable derivative, pharmaceutically acceptable polymorph, pharmaceutically acceptable prodrug, or pharmaceutically acceptable complex thereof.

12. A pharmaceutical solid oral sprinkle composition according claim 1, wherein the second antiretroviral drug comprises lopinavir.

13. A pharmaceutical solid oral sprinkle composition according to claim 1 for geriatric patients, comprising ritonavir and lopinavir.

14. A pharmaceutical solid oral sprinkle composition according to claim 1 for paediatric patients, comprising ritonavir and lopinavir.

15. A pharmaceutical solid oral sprinkle composition according to claim 1, comprising ritonavir in an amount from about 10 mg to about 200 mg.

16. A pharmaceutical solid oral sprinkle composition according to claim 12, comprising lopinavir in an amount from about 40 mg to about 800 mg.

17. A pharmaceutical solid oral sprinkle composition according to claim 1, wherein each particle comprises the first antiretroviral drug, the second antiretroviral drug and the at least one polymer.

18. A pharmaceutical solid oral sprinkle composition according to claim 1, wherein the first antiretroviral drug and the second antiretroviral drug are disposed in separate particles.

19. A pharmaceutical solid oral sprinkle composition according to claim 17, wherein the at least one polymer is disposed in the particle comprising the first antiretroviral drug.

20. A pharmaceutical solid oral sprinkle composition according to claim 17, wherein the at least one polymer is disposed in the particle comprising the second antiretroviral drug.

21. A pharmaceutical solid oral sprinkle composition form according to claim 1, wherein the at least one polymer comprises a water insoluble polymer.

22. A pharmaceutical solid oral sprinkle composition form according to claim 21, wherein the water insoluble polymer comprises: an acrylic copolymer; a polyvinylacetate; a cellulose derivative, such as ethylcellulose or cellulose acetate; or any combination thereof.

23. A pharmaceutical solid oral sprinkle composition form according to claim 1, wherein the at least one polymer comprises a water soluble polymer.

24. A pharmaceutical solid oral sprinkle composition according to claim 23, wherein the water soluble polymer comprises: copovidone; a homopolymer of a N-vinyl lactam, such as N-vinyl pyrrolidine or N-vinyl pyrrolidone; a copolymer comprising a N-vinyl lactam, such as N-vinyl pyrrolidine or N-vinyl pyrrolidone; polyvinylpyrrolidone (PVP); a copolymer of PVP and vinyl acetate; a co-polymer of N-vinyl pyrrolidone and vinyl acetate or vinyl propionate; a cellulose ester; a cellulose ether; a high molecular weight polyalkylene oxide, such as polyethylene oxide, polypropylene oxide, or a co-polymer of ethylene oxide and propylene oxide; or any combination thereof.

25. A pharmaceutical solid oral sprinkle composition form according to claim 1, wherein the at least one polymer comprises a water swellable polymer.

26. A pharmaceutical solid oral sprinkle composition according to claim 25, wherein the water swellable polymer comprises: a polyethylene oxide; a poly (hydroxy alkyl methacrylate); a poly (vinyl) alcohol having a low acetal residue and which is cross-linked with glyoxal, formaldehyde or glutaraldehyde; a mixture of methyl cellulose, cross-linked agar and carboxymethyl cellulose; a an acidic carboxy polymer; a polyacrylamide; a cross-linked water swellable indene-maleic anhydride polymer; a polyacrylic acid; a starch graft copolymer; an acrylate polymer polysaccharide comprising a condensed glucose unit, such as diester cross-linked polyglucan; an ion exchange resin; a sodium starch glycolate; a croscarmellose sodium, or any combination thereof.

27. A pharmaceutical solid oral sprinkle composition according to claim 1, having a ratio of the ritonavir and the second antiretroviral drug to the polymer in the range of from about 1:1 to about 1:6 by weight.

28. A pharmaceutical solid oral sprinkle composition according to claim 1, wherein the composition has a taste-masking property.

29. A pharmaceutical solid oral sprinkle composition according to claim 1, wherein the plurality of particles are provided in a dosage form comprising: powders, powders for reconstitution, pellets, beads, mini-tablets, film coated tablets, film coated tablets MUPS, orally disintegrating MUPS, pills, micro-pellets, small tablet units, MUPS, disintegrating tablets, dispersible tablets, capsules, granules, effervescent granules, sachets or any combination thereof.

30. A pharmaceutical solid oral sprinkle composition according to claim 1 further comprising one or more excipients comprising plasticizers, fillers or diluents; surfactants; solubility enhancers; disintegrants; binders; lubricants; non-ionic solubilisers; glidants; or any combination thereof.

31. A pharmaceutical solid oral sprinkle composition comprising ritonavir and lopinavir; and at least one polymer comprising: a water soluble polymer; a water swellable polymer; a water insoluble polymer; or any combination thereof.

32. A pharmaceutical solid oral sprinkle composition according to claim 1, wherein the plurality of particles are provided with a film coat; provided with film coat and a seal coat external to the film coat; or provided with a seal coat and a film coat external to the seal coat.

33. A pharmaceutical solid oral sprinkle composition according to claim 1, wherein the plurality of particles are uncoated.

34. A kit comprising a pharmaceutical solid oral sprinkle composition according to claim 1, the kit further comprising instructions for administration.

35. A process for preparing a pharmaceutical solid oral sprinkle composition according to claim 1, comprising hot melt extruding the first and second antiretroviral drugs to form an extrudate, then formulating the extrudate into the plurality of particles, and combining the plurality of particles to provide the solid oral composition.

36. A method according to claim 35, wherein the first and second antiretroviral drugs are mixed with the at least one polymer prior the hot melt extrusion step.

37. A method according to claim 35, comprising preparing a substantially homogeneous melt of the first and second antiretroviral drugs and optionally one or more excipient, extruding the melt, and cooling the melt until it solidifies, wherein the melt is preferably formed at a temperature from substantially 50° C. to substantially 200° C., and wherein the cooled extruded melt is preferably processed into said plurality of particles.

38. A method according to claim 35, wherein the first and second antiretroviral drugs, the at least one polymer, and optionally one or more excipient are processed to form a powder blend, which is transferred through the heated barrel of an extruder, whereby the powder blend melts and a molten solution product is formed, which is allowed to cool to form the extrudate.

39. A process for preparing a pharmaceutical solid oral sprinkle composition which is a sprinkle formulation comprising a plurality of particles, the plurality of particles comprising a first and a second antiretroviral drug, wherein the first antiretroviral drug comprises ritonavir, the process comprising:
(a) melt granulating one or more solubility enhancers and one or more pharmaceutically acceptable excipients with the or each drugs in purified water to form a granulated material;
(b) sieving the granulated material;
(c) drying the sieved granulated material to form dried granules;
(d) lubricating the dried granules with one or more lubricants and one or more second pharmaceutically acceptable excipients; and
(e) optionally further processing the lubricated dried granules to provide the dosage form.

40. A method of treatment of HIV infection or AIDS by administering a pharmaceutical solid oral sprinkle composition according to claim 1 to a patient in need thereof.

41. A pharmaceutical solid oral sprinkle composition according to claim 1 for use in the treatment of an HIV infection or AIDS.

42. A use of a pharmaceutical solid oral composition according to claim 1 in the manufacture of a medicament for the treatment of an HIV infection or AIDS.

43-44. (canceled)
